(19) United States
(12) Patent Application Publication
(10) Pub. No.: US 2006/0094674 A1
(43) Pub. Date: May 4, 2006

(54) Combination of mtor inhibitor and a tyrosine kinase inhibitor for the treatment of neoplasms

(76) Inventors: Benjamin G Neel, Wayland, MA (US); Golam Mohi, Newton, MA (US)

Correspondence Address: CLARK & ELBING LLP, 101 FEDERAL STREET, BOSTON, MA, 02110, US

(21) Appl. No.: 10/520,225

(22) PCT Filed: Jul. 3, 2003
(86) PCT No.: PCT/US03/20972

§ 371 Date: Nov. 10, 2005

Related U.S. Application Data
(60) Provisional application No. 60/394,029, filed on Jul. 5, 2002.
(60) Provisional application No. 60/412,402, filed on Sep. 20, 2002.

(51) Int. Cl. A61K 48/00; A61K 31/4745; A61K 39/395; A61K 49/00

(52) U.S. Cl. 514/44; 424/155.1; 514/291; 424/9.1

(57) ABSTRACT

The invention features methods and compositions including an mTOR inhibitor and a tyrosine kinase inhibitor for reducing the proliferation of and enhancing the apoptosis of neoplastic cells. The addition of an MEK inhibitor to this combination further enhances the effectiveness of this therapeutic method.

### STATEMENT AS TO FEDERALLY SPONSORED RESEARCH

This research has been sponsored by NIH Grants R01 DK50654 and. P01 DK50693. The U.S. government has certain rights to the invention.

### BACKGROUND OF THE INVENTION

The present invention relates to pharmaceutical combinations and their use in the treatment of disorders associated with the proliferation of neoplasms.

Cellular signal transduction is a fundamental mechanism whereby external stimuli that regulate diverse cellular processes are relayed to the interior of cells. Growth factor receptors (“GFRs”) are an important part of the signal transduction pathway. GFRs are cell-surface proteins. When bound by a growth factor ligand, GFRs are converted to an active form which interacts with proteins on the inner surface of a cell membrane. As the result of this interaction, one of the key biochemical mechanisms of signal transduction is initiated; i.e., the reversible phosphorylation of various proteins within the cell. Protein kinases (“PKs”) are enzymes that catalyze the phosphorylation of hydroxy groups on tyrosine, serine and threonine residues of proteins. This phosphorylation of intra-cellular proteins causes the formation of complexes with a variety of cytoplasmic signaling molecules that, in turn, effect numerous cellular responses such as cell division (proliferation), cell differentiation, cell growth, expression of metabolic effects to the extracellular microenvironment, among others (see Schlessinger and Ullrich, Neuron 9:303-391 (1992); Posada and Cooper, Mol. Biol. Cell. 3:583-392 (1992); and Hardie, Symp. Soc. Exp. Biol. 44:241-255 (1990)).

Growth factor receptors with tyrosine PK activity are known as receptor tyrosine kinases (“RTKs”). They comprise a large family of transmembrane receptors with diverse biological activity. At present, at least nineteen (19) distinct subfamilies of RTKs have been identified, including the EGFR, epithelial growth factor receptor, RTKs (HER1-4); the insulin receptor RIKs (IR, IGF-1R, and IRR); the PDGFR, platelet derived growth factor receptor, RTKs (PDGFR-α, PDGFR-β, CSFIE, Flt-3, c-kit and c-fms); the VEGFR, vascular endothelial growth factor receptor, RTKs (VEGF R1/flt-1, VEGF R2/KDR/FLK-1, VEGF R3/flt-4); the Trk, tropomyosin receptor kinases (TrkA, TrkB, and TrkC); and the FGF, fibroblast growth factor, RTKs (FGFR1-4), among others.

In addition to the RTKs, there also exists a family of entirely intracellular PTKs or cellular tyrosine kinases (“CTKs”). At present, over 24 CTKs in 11 subfamilies (Src, Frk, Btk, Csk, Abl, Zap70, Fes, Fps, Fak, Jak, Pkc, and Ack) have been identified. The Src subfamily appear so far to be the largest group of CTKs and includes Src, Yes, Fyn, Lyn, Lck, Blk, Hck, Fgr and Yrk, among others. For a more detailed discussion of CTKs see Bolen, Oncogene 8:2025-2031 (1993).

In addition to those listed above, there are tyrosine kinases that result from gene mutations. Examples include the BRC/ABL and TEL/ABL fusion genes, which encode for cytoplasmic proteins having constitutively active tyrosine kinase.

PTKs play an important role in the control of cellular processes including proliferation, differentiation, migration and survival. Enhanced PTK activity due to activating mutations or overexpression has been implicated in many human cancers. Malignant cell growth results from a breakdown in the mechanisms that control cell division and/or differentiation. It has been shown that the protein products of a number of proto-oncogenes are involved in the signal transduction pathways that regulate cell growth and differentiation. These protein products of proto-oncogenes include the extracellular growth factors, transmembrane growth factor PTK receptors (RTKs) and cellular PTKs (CTKs) discussed above. For example, EGFR is mutated and/or overexpressed in a variety of cancers, including brain, lung, squamous cell, bladder, gastric, breast, head and neck, oesophageal, gynecological and thyroid tumors.

Accordingly, it has been recognized that inhibitors of protein kinases are useful as selective inhibitors of the growth of mammalian cancer cells. Cancer therapy directed at specific and frequently occurring molecular alterations in signaling pathways of cancer cells has been validated through the clinical development and regulatory approval of agents such as Herceptin™, which targets HER-2 receptor, for the treatment of advanced breast cancer and Imatinib (Gleevec™), which targets the constitutively active tyrosine kinase BCR/ABL, for chronic myelogenous leukemia (CML) and Kit for gastrointestinal stromal tumors (GIST). See, for example, Shawver et al., Cancer Cell 1(2):117-123 10 (2002).

The macrolide fungicide rapamycin, a natural product with anti-tumor properties, is also capable of inhibiting signal transduction pathways that are necessary for the proliferation of cells. Rapamycin binds intracellularly to the immuunophilin FK506 binding protein 12 (FKBP12), and the resultant complex inhibits the serine protein kinase activity of mammalian target of rapamycin (mTOR). The inhibition of mTOR, in turn, blocks signals to at least two separate downstream pathways which control the translation of specific mRNAs required for cell proliferation.

Although Imatinib represents a promising therapy for CML, it often does not provide a cure and, in some instances, the development of resistance complicates the therapy. A number of Imatinib resistant BCR/ABL positive cell lines have been described (see, for example, Mahon, F. X., et al., Blood 96(3):1070-1079 (2000)) and resistance to Imatinib has been demonstrated in a nude mouse model (Gambacorti-Passerini, C., et al., J. Natl. Cancer inst. 25 92(20):1641-1650 (2000)). In addition, CML progression is accompanied by secondary genetic alterations (Ahuja, H., et al., J. Invest. 78(6):2042-2047 (1991); and Honda, H., et al., Blood 95(4):1144-1150 (2000)); thus survival of late stage CML leukemia cells may no longer be dependent on BCR/ABL tyrosine kinase activity. Imatinib induced hematological responses have been less dramatic in blast crisis patients compared to what is observed in chronic phase patients (Druker, B. J., et al., N. Engl. J Med. 344(14):1038-1042 (2001)). Recently, reactivation of BCR/ABL signaling either through mutation or amplification of BCR/ABL has been observed in patients that initially responded to Imatinib but then relapsed (Gorre, M. E., et al., Science 21:21 (2001); Barthe, C., et al., Science 293(5538):2163 (2001); and Hochhaus, A., et al., Science 293(5538):2163 (2001)). Finally, there have been no reports of patients undergoing Imatinib therapy becoming negative for the BCR/ABL translocation, suggesting the current therapy is not curative.

Additional therapies are needed to effectively eradicate cancers which are treated with signal transduction inhibitors that target tyrosine kinases, such as BCR/ABL positive leukemia.

### SUMMARY OF THE INVENTION

We have discovered that the combination of an mTOR inhibitor and a tyrosine kinase inhibitor is more effective than mTOR inhibitor monotherapy or tyrosine kinase inhibitor monotherapy for reducing the proliferation of and enhancing the apoptosis of cancer cells. The addition of an MEK (mitogen-activated protein kinase or extracellular signal-regulated kinase kinase) inhibitor to this combination further enhances the effectiveness of this combination therapy.

The present invention provides a method of treating a neoplasm in an individual in need thereof including administering to the patient at least one mTOR inhibitor in combination or in parallel with at least one tyrosine kinase inhibitor in amounts effective to treat the neoplasm.

Examples of mTOR inhibitors include, without limitation, any of the rapamycin macrolides described herein. Desirably, the mTOR inhibitor is a rapamycin macrolide selected from rapamycin, CCI-779, Everolimus, and ABT-578.

Tyrosine kinase inhibitors of the invention include small molecule inhibitors, tyrosine kinase antibodies, antisense oligomers, and RNAi inhibitors. Examples of tyrosine kinase inhibitors include any of those described herein. Desirable small molecule inhibitors include Imatinib, SU101, ZD1839, OSI-774, CI-1033, SU5416, SU6668, ZD4190, ZD6474, PTK787, PKI166, GW2016, EKB-509, EKB-569, CEP-701, CEP-751, PKC412, SU11248, and MLN518. Desirable tyrosine kinase antibodies include trastuzumab, C225, rhu-Mab VEGF, MDX-H210, 2C4, MDX-447, IMC-1C11, EMD 72000, RH3, and ABX-EGF.

In another aspect, the invention features a method of treating leukemia in a patient in need thereof including administering rapamycin to the patient in amounts effective to treat the leukemia.

Cancers to be treated using the methods of the invention include, without limitation, carcinoma of the bladder, breast, colon, kidney, liver, lung, head and neck, gall-bladder, ovary, pancreas, stomach, cervix, thyroid, prostate, or skin; a hematopoietic tumor of lymphoid lineage (i.e. leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); a hematopoietic tumor of myeloid lineage (i.e. acute myelogenous leukemia, chronic myelogenous leukemia, multiple myelogenous leukemia, myelodysplastic syndrome and promyelocytic leukemia); a tumor of mesenchymal origin (i.e. fibrosarcoma and rhabdomyosarcoma); a tumor of the central or peripheral nervous system (i.e. astrocytoma, neuroblastoma, glioma and schwannomas); melanoma; seminoma; teratocarcinoma; osteosarcoma; thyroid follicular cancer; and Kaposi's sarcoma.

Any of the methods described herein can be used to treat a neoplasm having cells characterized by abnormally high levels of tyrosine kinase activity.

The abnormally high tyrosine kinase activity can be epithelial growth factor receptor (EGFR) kinase activity. Abnormally high EGFR activity can be characteristic of non-small-cell lung cancers, breast cancers, ovarian cancers, bladder cancers, prostate cancers, salivary gland cancers, pancreatic cancers, endometrial cancers, colorectal cancers, kidney cancers, head and neck cancers, and glioblastoma multiforme. Using the methods of the present invention, a tyrosine kinase inhibitor targeted to EGFR can be used for the treatment of a cancers having abnormally high EGFR kinase activity. These include, but are not limited to, SU101, ZD1839, OSI-774, CI-1033, PKI166, GW2016, EKB-509, EKB-569, trastuzumab, C225, MDX-H210, 2C4, MDX-447, EMD 72000, RH3 and ABX-EGF. In one embodiment, cancers having abnormally high EGFR kinase activity are treated by administering any one of the above-listed tyrosine kinase inhibitors together or in parallel with a rapamycin macrolide selected from rapamycin, CCI-779, Everolimus, and ABT-578. Desirably, the tyrosine kinase inhibitor targeted to EGFR is selected from trastuzumab, C225, and ZD1839.

The abnormally high tyrosine kinase activity can be human epidermal growth factor receptor-2 (HER2/ERB2) activity. Abnormally high HER2 activity can be characteristic of breast cancer, ovarian cancer, bladder cancer, salivary gland cancer, endometrial cancer, pancreatic cancer, and non-small-cell lung cancer. Using the methods of the present invention, a tyrosine kinase inhibitor targeted to HER2 can be used for the treatment of a cancers having abnormally high HER2 activity. These include, but are not limited to, CI-1033, GW2016, trastuzumab, MDX-H210, MDX-447, ABX-EGF, and 2C4. In one embodiment, cancers having abnormally high HER2 activity are treated by administering any one of the above-listed tyrosine kinase inhibitors together or in parallel with a rapamycin macrolide selected from rapamycin, CCI-779, Everolimus, and ABT-578. Desirably, the tyrosine kinase inhibitor targeted to HER2 is trastuzumab.

The abnormally high tyrosine kinase activity can be platelet derived growth factor receptor (PDGFR) kinase activity. Abnormally high PDGFR activity can be characteristic of gastrointestinal stromal tumor, small cell lung cancer, glioblastoma multiforme, and prostate cancer. Using the methods of the present invention, a tyrosine kinase inhibitor targeted to PDGFR can be used for the treatment of a cancers having abnormally high PDGFR kinase activity. These include, but are not limited to Imatinib, SU101, MLN518, and PTK787. In one embodiment, cancers having abnormally high PDGFR kinase activity are treated by administering any one of the above-listed tyrosine kinase inhibitors together or in parallel with a rapamycin macrolide selected from rapamycin, CCI-779, Everolimus, and ABT-578. Desirably, the tyrosine kinase inhibitor targeted to PDGFR is Imatinib.

The abnormally high tyrosine kinase activity can be Flt-3 activity. Abnormally high Flt-3 activity can be characteristic of acute myeloid leukemia. Using the methods of the present invention, a tyrosine kinase inhibitor targeted to Flt-3 can be used for the treatment of a cancers having abnormally high Flt-3 kinase activity. These include, but are not limited to MLN518, SU11248, and PKC412. In one embodiment, cancers having abnormally high Flt-3 kinase activity are treated by administering any one of the above-listed tyrosine kinase inhibitors together or in parallel with a rapamycin macrolide selected from rapamycin, CCI-779, Everolimus, and ABT-578. Desirably, the tyrosine kinase inhibitor targeted to Flt-3 is PKC412.

The abnormally high tyrosine kinase activity can be tropomyosin receptor kinase (Trk) activity. Abnormally high Trk activity can be characteristic of prostate cancer and pancreatic cancer. Using the methods of the present invention, a tyrosine kinase inhibitor targeted to Trk can be used for the treatment of a cancers having abnormally high Trk kinase activity. These include, but are not limited to CEP701 or CEP705. In one embodiment, cancers having abnormally high Trk kinase activity are treated by administering any one of the above-listed tyrosine kinase inhibitors together or in parallel with a rapamycin macrolide selected from rapamycin, CCI-779, Everolimus, and ABT-578.

Abnormally high vascular endothelial growth factor receptor (VEGFR) kinase activity is not typically found in the cells of a neoplasm, but is often found in the endothelial cells which vascularize the neoplasm. Thus, vascular endothelial growth factor receptor (VEGFR) kinase activity is a useful target for most solid tumors. Using the methods of the present invention, a tyrosine kinase inhibitor targeted to VEGFR can be used for the treatment of solid tumors. These include, but are not limited to SU5416, SU6668, ZD4190, ZD6474, PTK787, IMC-1C11, and rhu-Mab VEGF. In one embodiment, cancers having abnormally high VEGFR kinase activity are treated by administering any one of the above-listed tyrosine kinase inhibitors together or in parallel with a rapamycin macrolide selected from rapamycin, CCI-779, Everolimus, and ABT-578.

The abnormally high tyrosine kinase activity can be constitutively active tyrosine kinase BCR/ABL. BCR/ABL kinase activity can be characteristic of chronic myelogenous leukemia (CML). Using the methods of the invention, CML patients are treated, for example, with a combination of a rapamycin macrolide selected from rapamycin, CCI-779, Everolimus, and ABT-578 and a tyrosine kinase inhibitor which is active against BCR/ABL. Desirably, the kinase inhibitor which is active against BCR/ABL is Imatinib.

In any of the above methods, an MEK inhibitor can be included to enhance the effectiveness of the combination therapy. Examples of MEK inhibitors include any of those described herein. In one embodiment, the MEK inhibitor is selected from the group consisting of PD184352, PD198306, PD98059, UO126, Ro092210, and L783277. For example, an mTOR inhibitor, Imatinib, and UO126, an MEK inhibitor, can be used to treat CML or GIST.

Any of the combinations described herein can be used to treat a neoplasm which is resistant to monotherapy using the tyrosine kinase inhibitor of the combination. For example, the combination of an mTOR inhibitor and Imatinib, or the combination of an mTOR inhibitor and Imatinib and MEK inhibitor, can be used to treat Imatinib-resistant neoplasms. In one embodiment, the combination of an mTOR inhibitor and Imatinib can be used to treat Imatinib-resistant CML and GIST. In another example, the combination of mTOR inhibitor and PCK412, or the combination of an mTOR inhibitor and PCK412 and MEK inhibitor, can be used to treat PCK412-resistant neoplasms. In one embodiment, the combination of an mTOR inhibitor and PCK412 can be used to treat PCK412-resistant acute myeloid leukemia.

For any of the above methods, the mTOR and the tyrosine kinase inhibitors, or the mTOR, tyrosine kinase, and MEK inhibitors, can be administered in parallel within 30 days of each other. Desirably, the mTOR and tyrosine kinase inhibitors are administered in parallel within five days of each other, 24 hours of each other, simultaneously, or they are administered together. For the three component therapeutic combinations including mTOR, tyrosine kinase, and MEK inhibitors, each component is, desirably, administered in parallel within five days of another component, 24 hours of another component, or all three are administered simultaneously, or together.

For any of the combinations described herein, the invention also features a method of determining whether a neoplasm in a human patient responds to a combination including an mTOR inhibitor and tyrosine kinase inhibitor. This method includes the steps of (a) administering the combination to the human patient; and (b) monitoring the patient to determine whether the neoplasm responds to the combination. Optionally, the combination includes an MEK inhibitor. This method can be performed, for example, to determine whether the combination has enhanced efficacy in comparison to monotherapy using any one of the inhibitors in the combination. This method can also be used to determine which regimens are effective for treating the neoplasm (e.g., variables include the amount of each inhibitor in the combination, routes of administration for each inhibitor, and/or the intervals between administrations). This method can also be used to determine which types of neoplasms respond to the combination therapy.

Administration of the mTOR, tyrosine kinase, and MEK inhibitors can be achieved by a variety of routes, such as by parenteral routes (e.g., intravenous, intraarterial, intramuscular subcutaneous injection), topical, inhalation (e.g., intrabronchial, intranasal or oral inhalation or intranasal drops), oral, rectal, or other routes.

The present invention features a pharmaceutical pack including a rapamycin macrolide and a tyrosine kinase inhibitor. Desirably, the rapamycin macrolide and the tyrosine kinase inhibitor are formulated separately and in individual dosage amounts. The pharmaceutical pack may further include an MEK inhibitor.

The present invention also features a pharmaceutical composition including an effective amount of a rapamycin macrolide and a tyrosine kinase inhibitor, together with a pharmaceutically acceptable carrier or diluent. The pharmaceutical composition may further include an MEK inhibitor.

Compounds useful in the present invention include those described herein in any of their pharmaceutically acceptable forms, including isomers such as diastereomers and enantiomers, salts, and solvates thereof, as well as racemic mixtures of the compounds described herein.

By “treating” is meant to slow the spreading of the cancer, to slow the cancer's growth, to kill or arrest cancer cells that may have spread to other parts of the body from the original tumor, to relieve symptoms caused by the cancer, or to prevent cancer. The symptoms to be relieved using the combination therapies described herein include pain, and other types of discomfort.

The terms “administration” and “administering” refer to a method of giving a dosage of a pharmaceutical composition to a patient, where the method is, e.g., topical, oral, intravenous, intraperitoneal, or intramuscular. The preferred method of administration can vary depending on various factors, e.g., the components of the pharmaceutical composition, site of the potential or actual disease and severity of disease.

By administration “in parallel” is meant that the mTOR inhibitor, tyrosine kinase inhibitor, and, optionally, the MEK inhibitor are formulated separately and administered separately.

By administered “together” is meant that the mTOR inhibitor, tyrosine kinase inhibitor, and, optionally, the MEK inhibitor are formulated together in a single pharmaceutical composition and administered together.

By “effective amount” is meant the amount of a compound required to treat a neoplasm. The effective amount of mTOR inhibitor, tyrosine kinase inhibitor, and, optionally, the MEK inhibitor used to practice the present invention for the treatment of a neoplasm varies depending upon the manner of administration, the age, body weight, and general health of the subject. Ultimately, the attending physician, will decide the appropriate amount and dosage regimen. Such amount is referred to as an “effective” amount.

As used herein, “individual” or “patient” includes humans, cattle, pigs, sheep, horses, dogs, and cats, and also includes other vertebrates, most preferably, mammalian species.

By “rapamycin macrolide” is meant naturally occurring forms of rapamycin in addition to rapamycin analogs and derivatives which target and inhibit mTOR.

By “tyrosine kinase inhibitor” is meant a molecule that inhibits the function or the production of one or more tyrosine kinases. Tyrosine kinase inhibitors include small molecule inhibitors of tyrosine kinases, antibodies to tyrosine kinases, and antisense oligomers and RNAi inhibitors that reduce the expression of tyrosine kinases.

By “small molecule” inhibitor is meant a molecule of less than about 3,000 daltons having tyrosine kinase antagonist activity.

By “antisense” or “antisense oligomer” is meant any oligonucleotide or oligonucleoside that acts to inhibit the expression or function of a tyrosine kinase.

By “RNAi inhibitor” is meant any double stranded RNA that acts to inhibit the expression or function of a tyrosine kinase (for an example of RNAi technology, see Zamore et al., Cell 101:25-33 (2000)).

By “abnormally high levels of kinase activity” is meant an increase in tyrosine kinase activity associated with malignant cell growth. An increase in tyrosine kinase activity can result from overexpression or mutation of a kinase gene. For example, the BCR/ABL mutation encodes a cytoplasmic protein with aberrant constitutive tyrosine kinase activity, resulting in uncontrolled proliferation.

As used herein, “resistance” or “resistant” refers to a neoplasm having cells that express a resistant mutant form of the tyrosine kinase or cells that overexpress the tyrosine kinase targeted by the tyrosine kinase inhibitor used in the combination therapy described herein. Resistance includes other known mechanisms of resistance (e.g., efflux pump in resistant cells). The net effect of the resistance is that the use of the tyrosine kinase inhibitor as a monotherapy for the treatment of the resistant cells is less effective than when used to treat a non-resistant cells.

As used herein, a neoplasm “responds” to a combination of mTOR inhibitor, tyrosine kinase inhibitor, and, optionally, MEK inhibitor, if the spread of the neoplasm is slowed, if the growth of the neoplasm is slowed, if neoplasm cells spreading from the site of origin to other parts of the body are killed or arrested, or if the combination relieves symptoms caused by the neoplasm. The symptoms relieved when a neoplasm responds to the combination therapies described herein include pain, and other types of discomfort.

As used herein, “monitoring” a patient to determine whether the neoplasm responds to the combination therapy includes any established protocol for monitoring the progression of a neoplastic disorder. Monitoring can include, for example, the use of biopsies, use of surrogate markers (e.g., PSA levels in prostate cancer patients), and the use of imaging techniques (e.g., CT scans, bone scans, chest x-rays, MRI scans) to see if the neoplasm has grown or spread, among other protocols.

The invention provides methods of treating neoplasms associated with enhanced tyrosine kinase activity, allowing for improved cancer therapy while permitting lower doses of mTOR, tyrosine kinase, and, optionally, MEK inhibitors to be used. Other features and advantages of the invention will be apparent from the following detailed description, and from the claims.

### BRIEF DESCRIPTION OF THE DRAWINGS

FIG. 1A is a graph illustrating the effects of rapamycin (R) on BCR-ABL-transformed primary B lymphoblasts.

FIG. 1B is a graph illustrating the effects of rapamycin (R) and Imatinib (I) on BCR-ABL-transformed primary B lymphoblasts.

FIG. 2A is a graph illustrating the effects of Imatinib (I) and rapamycin (R) on BCR/ABL-evoked myeloid colony outgrowth.

FIG. 2B is a graph illustrating the effects of Imatinib (I) and rapamycin (R) on K562 cells derived from a blast crisis CML patient.

FIG. 3A is a graph illustrating the inhibitory effects of Imatinib (I) and rapamycin (R) in Ba/F3 cells expressing wild type BCR/ABL.

FIG. 3B is a graph illustrating the inhibitory effects of Imatinib (I) and rapamycin (R) in Ba/F3 cells expressing Imatinib-resistant BCR/ABL.

FIG. 4A is a picture of an immunoblotting assay illustrating the inhibitory effects of Imatinib (I) and rapamycin (R) in Ba/F-BCR/ABL WT and Imatinib-resistant Ba/F-BCR/ABL T3151 cells on the activation of p70 S6K, Erk1/2 kinases.

FIG. 4B is a picture of an immunoblotting assay illustrating the inhibitory effects of Imatinib (I) and rapamycin (R) in Imatinib-resistant Ba/F-BCR/ABL T315I cells on the phosphorylation of 4E-BP1.

FIG. 5A is a graph illustrating the effects of PKC412 (P) and rapamycin (R) on the proliferation of PKC412-sensitive Ba/F-FLT3-ITD cells.

FIG. 5B is a graph illustrating the effects of PKC412 (P) and rapamycin (R) on the proliferation of PKC412-resistant Ba/F-FLT3-ITD F691I cells.

FIG. 5C is a picture of an immunoblotting assay illustrating the inhibitory effects of PKC412 (P) and rapamycin (R) in PKC412-sensitive Ba/F-FLT3-ITD cells on the activation of p70 S6K kinases.

FIG. 5D is a picture of an immunoblotting assay illustrating the inhibitory effects of PKC412 (P) and rapamycin (R) in PKC412-sensitive Ba/F-FLT3-ITD cells on the activation of 4E-BP1 kinases.

FIG. 6A is a graph illustrating the effects of Imatinib (I) or rapamycin (R) or UO126 (UO) alone or in various combinations, as indicated, on the proliferation of bone marrow cells expressing BCR/ABL.

FIG. 6B is a graph illustrating the effects of Imatinib (I) or rapamycin (R) or UO126 (UO) alone or in various combinations, as indicated, on the proliferation of Imatinib-resistant Ba/F-BCR/ABL T315I cells.

FIG. 6C is a graph illustrating the effects of PKC412 (P) or rapamycin (R) or UO126 (UO) alone or in various combinations, as indicated, on the proliferation of PKC412-resistant Ba/F-FLT3-ITD F691I cells.

FIG. 7 is a graph illustrating the effect of Herceptin and rapamycin (RAPA) on proliferation of MCF-7 cells.

FIG. 8 is a graph illustrating the effect of Herceptin and rapamycin (RAPA) on proliferation of SKBR3 cells.

FIG. 9 is a graph illustrating the effects of Imatinib and rapamycin (RAPA) on CWR-22 cells.

FIG. 10 is a graph illustrating the effects of Imatinib and rapamycin (RAPA) on LnCap cells.

FIG. 11 is a graph illustrating the effect of Imatinib (I) and rapamycin (R) on survival rates in CML tumor bearing mice.

### DETAILED DESCRIPTION OF THE INVENTION

We have discovered that the combination of an mTOR and tyrosine kinase inhibitors, or mTOR, tyrosine kinase, and MEK inhibitors, is more effective than rapamycin macrolide monotherapy or tyrosine kinase inhibitor monotherapy for reducing the proliferation of and increasing the apoptosis of cancer cells.

Rapamycin Macrolides

Rapamycin (Sirolimus) is an immunosuppressive lactam macrolide that is produced by Streptomyces hygroscopicus, and which has the structure depicted in Formula I. See, for example, McAlpine, J. B., et al., J. Antibiotics 44: 688 (1991); Schreiber, S. L., et al., J. Am. Chem. Soc. 113: 7433 (1991); and U.S. Pat. No. 3,929,992, incorporated herein by reference.

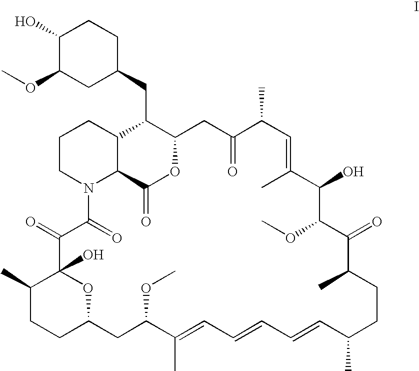

Wherever the present application refers to “rapamycin macrolide”, in addition to naturally occurring forms of rapamycin, the invention further includes rapamycin analogs and derivatives. Many such analogs and derivatives are known in the art. Examples include those compounds described in U.S. Pat. Nos. 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; all of which are incorporated herein by reference.

Desirable rapamycin macrolides for use in the present methods include rapamycin, CCI-779, Everolimus (also known as RAD001), and ABT-578. CCI-779 is an ester of rapamycin (42-ester with 3-hydroxy-2-hydroxymethyl-2-methylpropionic acid), disclosed in U.S. Pat. No. 5,362,718. Everolimus is an alkylated rapamycin (40-O-(2-hydroxyethyl)-rapamycin, disclosed in U.S. Pat. No. 5,665,772.

Tyrosine Kinase Inhibitors

Any tyrosine kinase inhibitor can be used in the methods of the present invention. These include small molecule inhibitors, antibodies to tyrosine kinase, antisense oligomers, and RNAi inhibitors.

The tyrosine kinase inhibitor used in the methods of the invention will be selected based upon the type of cancer being treated. Specifically, the inhibitor is selected based upon which tyrosine kinases exhibit abnormally high levels of activity characteristic of the cancer to be treated. For example, the BCR/ABL fusion protein occurs in 95% of CML and 10-15% of acute lymphoblastic leukemia patients. Accordingly, using methods of the invention, CML patients can be treated, for example, with a combination of rapamycin and a tyrosine kinase inhibitor which is active against BCR/ABL.

Tyrosine kinase inhibitors and their respective targets are provided in Table 1 (small molecule inhibitors) and Table 2 (tyrosine kinase antibodies).

TABLE 1
Selected Small Molecule Tyrosine Kinase Inhibitors

| Drug | Company | Target (s) |
|---|---|---|
| Imatinib (Gleevec ™) | Novartis | PDGFR (c-kit), ab1, |
| SU101 (Leflunomide) | Pharmacia | PDGFR, EGFR |
| ZD1839 (Iressa ™) | AstraZeneca | EGFR (HER1) |
| OSI-774 (Tarceva ™) | Oncogene Science | EGFR (HER1) |
| CI-1033 | Pfizer | EGFR (HER1 and HER2) |
| SU5416 | Pharmacia | VEGFR, PDGFR |
| SU6668 | Pharmacia | VEGFR, PDGFR, FGFR |
| ZD4190 | AstraZeneca | VEGFR (KDR/Flt-1) |
| ZD6474 | AstraZeneca | VEGFR (KDR/Flt-1) |
| PTK787 | Novartis | VEGFR (KDR/Flt-1), |
| | | PDGFR, c-kit |
| PKI166 | Novartis | EGFR (HER1) |
| GW2016 | GlaxoSmithKline | EGFR (HER1 and HER2) |
| EKB-509 | Wyeth | EGFR (HER1) |
| EKB-569 | Wyeth | EGFR (HER1) |
| CEP-701 | Cephalon | Trk |
| CEP-751 | Cephalon | Trk |
| MLN518 | Millenium | Flt-3, PDGFR (c-kit) |
| SU11248 | Pharmacia | Flt-3 |
| PKC412 | Novartis | Flt-3 |

TABLE 2
Selected Antibody Tyrosine Kinase Inhibitors

| Drug | Company | Target(s) |
|---|---|---|
| trastuzumab (Herceptin ™) | Genentech | EGFR (HER2) |
| C225 (Erbitux ™) | ImClone | EGFR |
| rhu-Mab VEGF (Avastin ™) | Genentech | VEGFR |
| MDX-210 | Medarex | EGFR (HER2) |
| 2C4 | Genentech | EGFR (HER2) |
| MDX-447 | Medarex | EGFR |
| ABX-EGF | Abgenix | EGFR |
| EMD 72000 | Merck | EGFR |
| RH3 | York Medical | EGFR |
| IMC-1C11 | ImClone | VEGFR2 |

MEK Inhibitors

Any MEK inhibitor can be used in the methods of the present invention. MEK inhibitors can be identified using known MEK inhibition assays. For example, the assays described in U.S. Pat. No. 5,525,625 or in WO 02/06213 A1, can be used to identify MEK inhibitors. Examples of MEK inhibitors include those compounds described in U.S. Pat. Nos. 6,545,030, 6,506,798, 6,492,363, 6,469,004, 6,455,582, 6,440,966, 6,310,060, 6,214,851, and 5,525,625, and U.S. Publication Nos. US 2003/0092748 A1, US 2003/0078428 A1, US 2003/0045521 A1, US 2003/0004193 A1, and US 2002/0022647 A1.

The MEK inhibitor used in the methods of the invention will be combined with a rapamycin macrolide and a tyrosine kinase inhibitor selected based upon the type of cancer being treated. Specifically, the inhibitor is selected based upon which tyrosine kinases exhibit abnormally high levels of activity characteristic of the cancer to be treated. For example, the BCR/ABL fusion protein occurs in 95% of CML and 10-15% of acute lymphoblastic leukemia patients. Accordingly, using methods of the invention, CML patients can be treated, for example, with a combination of rapamycin macrolide, MEK inhibitor, and a tyrosine kinase inhibitor which is active against BCR/ABL.

Several MEK inhibitors which can be used to practice the methods described herein are provided in Table 3.

TABLE 3
Selected MEK Inhibitors

| Drug | Company |
|---|---|
| PD184352/CI-1044 | Pfizer |
| PD198306 | Pfizer |
| PD98059 | Pfizer |
| UO126 | Promega |
| Ro092210 | Roche |
| L783277 | Merck |

Therapy

The methods of the present invention can be used for the treatment of a variety of cancers. In particular, the present methods can be used for the treatment of CML. CML progresses through distinct clinical stages. The earliest stage, termed the chronic phase, is characterized by the expansion of terminally differentiated neutrophils. Over several years the disease progresses to an acute phase termed blast crisis, characterized by maturation arrest with excessive numbers of undifferentiated myeloid or lymphoid progenitor cells. The BCR-ABL oncogene is expressed at all stages, but blast crisis is characterized by multiple additional genetic and molecular changes. Once the patient enters the blast crisis phase of the disease there are few curative options available.

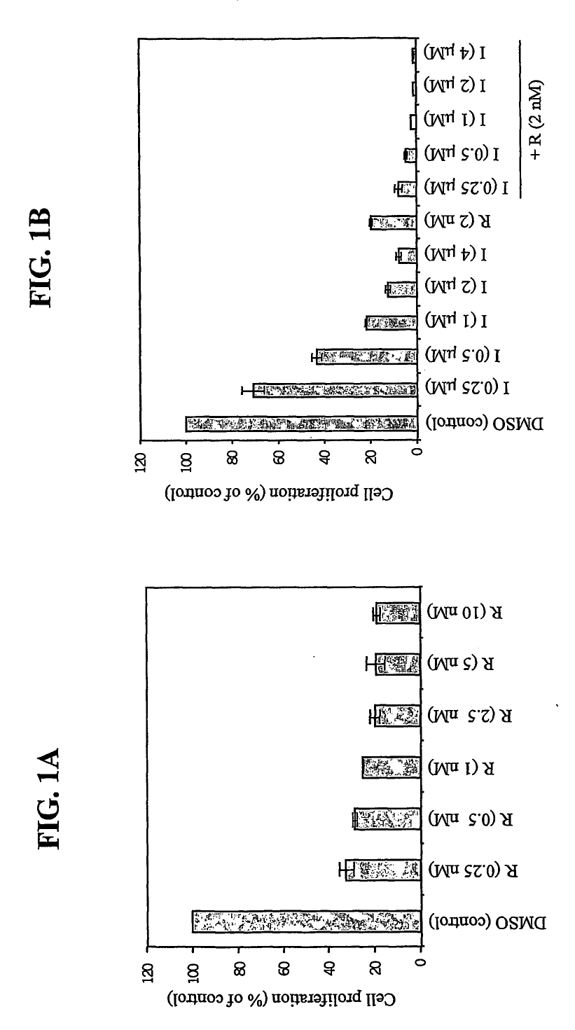

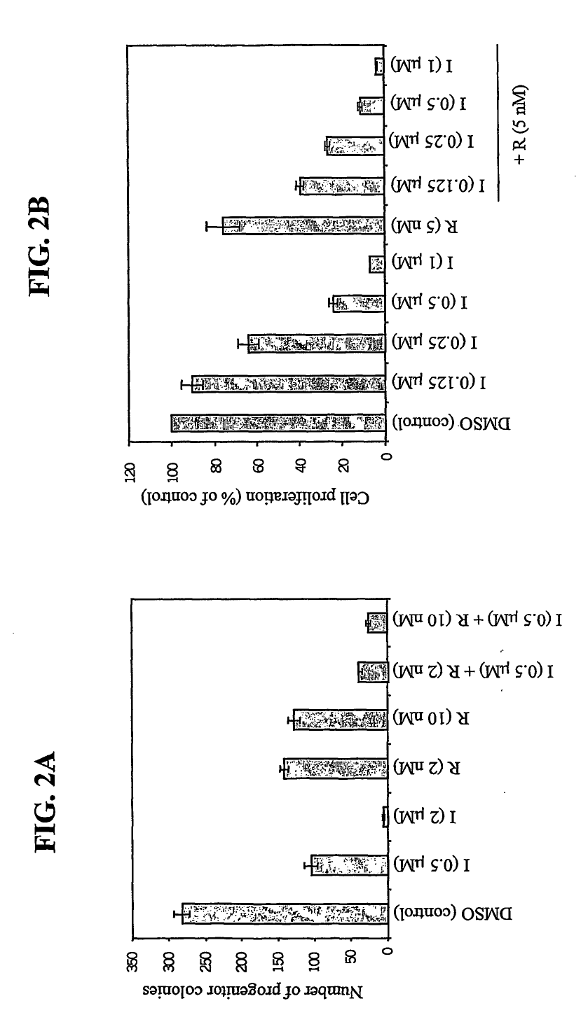

The present methods can be used to treat both the acute and chronic stages of CML. This is demonstrated in FIGS. 1A, 1B, 2A, and 2B; and Table 4. The combination of rapamycin and Imatinib is much more effective in preventing proliferation and colony formation than either drug used alone.

In the methods of the present invention, the dosage and frequency of administration of the mTOR, tyrosine kinase, and, optionally, MEK inhibitors can be controlled independently. For example, one compound may be administered orally three times per day, while the second compound may be administered intravenously once per day. The compounds may also be formulated together such that one administration delivers two, or even all three, of the compounds.

The exemplary dosage of mTOR, tyrosine kinase, and MEK inhibitor to be administered will depend on such variables as the type and extent of the disorder, the overall health status of the patient, the therapeutic index of the selected rapamycin macrolide and tyrosine kinase inhibitor, and their route of administration. Standard clinical trials maybe used to optimize the dose and dosing frequency for any particular combination of the invention.

Pharmaceutical Compositions

The invention features methods of treating cancer by administering an mTOR inhibitor, a tyrosine kinase inhibitor, and, optionally, an MEK inhibitor together or in parallel with each other. These may be formulated together or separately and administered to patients with a pharmaceutically acceptable diluent, carrier, or excipient, in unit dosage form. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (20th ed., ed. A. R. Gennaro A R., 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the mTOR inhibitor and/or tyrosine kinase inhibitor and/or MEK inhibitor. Nanoparticulate formulations (e.g., biodegradable nanoparticles, solid lipid nanoparticles, liposomes) may be used to control the biodistribution of the rapamycin macrolide and/or tyrosine kinase inhibitor. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the mTOR inhibitor, tyrosine kinase inhibitor, and, optionally, MEK inhbitor in the formulation will vary depending upon a number of factors, including the dosage of the drug to be administered, and the route of administration.

The mTOR inhibitor and/or tyrosine kinase inhibitor and/or MEK inhibitor may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of any of the mTOR inhibitor, tyrosine kinase inhibitor, or MEK inhibitor in controlled release formulations is useful where the inhibitor has (i) a narrow therapeutic index (e.g., the difference between the plasma concentration leading to harmful side effects or toxic reactions and the plasma concentration leading to a therapeutic effect is small; generally, the therapeutic index, Th, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the rapamycin macrolide and/or tyrosine kinase inhibitor. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Experimental Procedures

Cell Lines and Cell Culture

BTa/F3 cell lines expressing p210 BCR/ABL wild type (Ba/F-BCR/ABL WT), p210 BCR/ABL T315I (Ba/F-BCR/ABL T315I), FLT3-ITD (Ba/F-FLT3-ITD), FLT3-ITD F691I (Ba/F-FLT3-ITD F691I) were grown in RPMI 1640 with 10% (v/v) fetal calf serum (FCS) plus antibiotics (Penicillin/Streptomycin). BCR/ABL-transformed B-lymphoblasts were generated as described previously (Sattler et al., Cancer Cell 1:479 (2002)) and maintained in RPMI plus 20% FCS, antibiotics and 50 μM 2-mercaptoethanol (2-ME). K562 cells (ATCC number CCL-243) were cultured in RPMI supplemented with 10% FCS and antibiotics (Penicillin/Streptomycin).

Reagents

Imatinib (Novartis Pharmaceuticals, Basel, Switzerland), rapamycin (Sigma-Chemical Co., St. Louis, Mo.), PKC412 (Novartis), and UO126 (Calbiochem, La Jolla, Calif.) solutions were prepared in sterile DMSO and stored at −20 C.

Proliferation, Cell Cycle and Apoptosis Assays

BCR/ABL-transformed primary B-lymphoblasts (1×10⁴ cells/well) were cultured in 96-well plates in RPMI medium containing 20% FCS for 24 hours. Ba/F-BCR/ABL WT, Ba/F-BCR/ABL T315I, Ba/F-FLT3-ITD, Ba/F-FLT3-ITD F691I and K562 cells (3.5×10³ cells/well) were cultured in 96-well plates in RPMI containing 10% FCS for 48 or 60 hours. Cells were exposed to varying concentrations and combinations of drugs, as indicated. Four to six hours before harvesting, [³H]-thymidine (1 μCi/well) was added and [³H]-thymidine incorporation was determined using a Cell Harvester (Skatron, Sterling, Va.).

For cell cycle and apoptosis assays, live cells from randomly growing BCR/ABL-transformed B-lymphoblasts were isolated using Histopaque (Sigma). After washing twice, the cells were resuspended in regular growth medium (RPMI-containing 20% FCS and 50 μM 2-mercaptoethanol), exposed to inhibitors at the indicated concentrations for 24 hours, harvested and fixed in 70% ethanol, for 3 hours at −20 C. Fixed cells were stained with propidium iodide, and cell cycle parameters were analyzed by using FACScan and Modfit LT software.

Bone Marrow Transduction and Colony Formation Assays

A high-titer, helper virus-free retroviral stock of MSCV p210 (BCR/ABL)-IRES-GFP was prepared by transient transfection of 293T cells using the kat ecotropic packaging system (Million and Van Etten, Blood 96:664 (2000)). Bone marrow transduction and colony formation assays were performed as described previously (Sattler et al., Cancer Cell 1:479 (2002)). Briefly, bone marrow cells from mice were transduced with BCR/ABL-expressing retroviruses and then plated in triplicate in MethoCult M3234 medium (Stem Cell Technologies, Vancouver) at 1×10⁵ cells/35 mm dish in the presence or absence of different concentrations of drugs and maintained at 37° C., 5% CO₂. Myeloid colonies were scored at day 10.

Immunoblotting

Cells were lysed in a buffer containing Tris-HCl (50 mM, pH 8.0), NaCl (150 mM), NP40 (1% v/v), NaF (10 mM), 2 mM sodium orthovanadate and a cocktail of protease inhibitors, as described previously (Gu et al., Mol. Cell. Biol. 20:7109 (2000)). Cell lysates containing equivalent amounts of protein (50 g) were loaded and separated by SDS-PAGE. Immunoblotting was performed using phospho-specific antibodies reactive with Thr389 of p70 S6K, Ser65 of 4E-BP1 or Thr202/Tyr204 of p44/42 Erk (Cell Signaling Technology, Beverly, Ma.). Detection was by enhanced chemiluminescence (ECL). To control for equal loading, blots were reprobed with antibodies that detect total Erk2 or p70 S6K (Santa Cruz Biotechnology).

### Example 1

### Effects of Rapamycin and/or Imatinib on BCR-ABL-Transformed Primary B Lymphoblasts

BCR/ABL-transformed primary B lymphoblast cells were seeded in triplicate in 96 well plates at 1×10⁴ cells/well and exposed to various concentrations (0.25-10 nM) of rapamycin for 24 hours. As a control, cells were treated with DMSO vehicle for 24 hours. Cell proliferation was measured by [³H]-thymidine incorporation, expressed as the percentage of control (DMSO-treated) incorporation. The results are provided in FIG. 1A.

BCR/ABL-transformed primary B lymphoblasts were exposed to the indicated concentrations provided in FIG. 1B of Imatinib (0.25-4 μM) alone or in combination with rapamycin (2 nM) for 24 hours, followed by measurement of [³H]-thymidine incorporation. The results shown are representative of three independent experiments

Rapamycin inhibited the proliferation of these cells at doses significantly below typical serum levels (˜5-15 nM) achieved in transplant patients (MacDonald et al., Clin. Ther. 22 Suppl. B:B101(2000)) (see FIG. 1A). As expected, Imatinib also inhibited the proliferation of BCR/ABL-transformed primary B-lymphoblasts in a dose-dependent manner (FIG. 1B). Remarkably, combining rapamycin with Imatinib resulted in markedly decreased proliferation (FIG. 1B). Notably, the doses of each agent that, when used in combination, caused profound inhibition of cell proliferation were below the typical serum levels of the two drugs when used alone for anti-leukemic (Imatinib) or immunosuppressive (rapamycin) therapy (Druker et al., N. Engl. J. Med. 344:1038 (2002); MacDonald et al., Clin. Ther. 22 Suppl. B:B101(2000)).

### Example 2

### Effects of Rapamycin and/or Imatinib on BCR/ABL-Evoked Myeloid Colony Outgrowth and on K562 Cells Derived from a Blast Crisis CML Patient

Bone marrow (BM) cells from wild type (WT) mice were transduced with BCR/ABL-expressing retroviruses and plated in methylcellulose, in triplicate using MethoCult M3234 medium, in the absence of cytokines. As expected, BCR/ABL promoted cytokine-independent myeloid colony outgrowth (Gishizky and Witte, Science 256:836 (1992)). Rapamycin (2-10 nM or Imatinib (0.5 μM) alone inhibited myeloid colony formation by 50-60% (see FIG. 2A). However, combining these two agents resulted in greater than 90% decrease in BCR/ABL-induced myeloid colonies. This data shows that a combination of Imatinib and rapamycin may be more effective therapy for treatment of CML patients.

To ensure that the effects of the Imatinib/rapamycin combination were not restricted to murine cells, we carried out similar experiments on K562 cells, which are derived from a blast crisis CML patient (Lozzio and Lozzio, Blood 45:321.(1975)). Cells were exposed to the indicated concentrations (0.125-1 μM), as indicated in FIG. 2B, of Imatinib alone or in combination with rapamycin (5 nM) for 60 hours. Cell proliferation was measured by [³H]-thymidine incorporation, expressed as percentage of control (vehicle-treated) cells.

Even in this highly transformed cell line, rapamycin alone had some ability to inhibit [³H]-thymidine incorporation up to ˜25% inhibition at 5 nM (see FIG. 2B). Moreover, co-administration of rapamycin and Imatinib resulted in a dramatically increased inhibition. Together, these results suggest that combination therapy with these two approved drugs may have broad efficacy against BCR/ABL-transformed cells, and may have activity in CML blast crisis.

### Example 3

### Rapamycin Enhances the Growth Inhibitory Effects of Imatinib in Ba/F3 Cells Expressing Wild Type and Imatinib-Resistant BCR/ABL

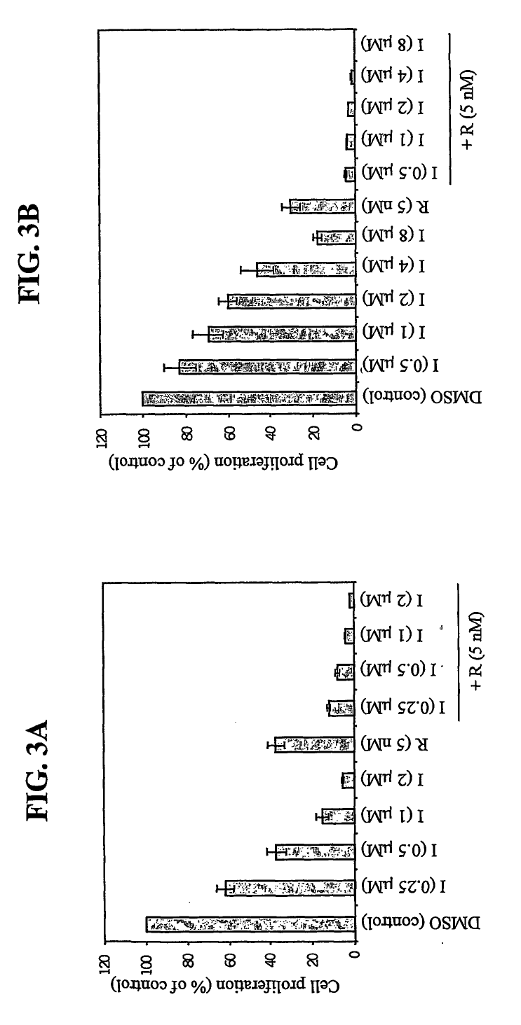

Imatinib resistance is an emerging clinical problem. Because rapamycin inhibits the proliferation of BCR/ABL-transformed lymphoid and myeloid cells, and rapamycin acts on a distinct downstream target of BCR/ABL, we tested whether rapamycin inhibited the proliferation of hematopoietic cells expressing Imatinib-resistant mutants of BCR/ABL. Ba/F-BCR/ABL WT (FIG. 3A) and Ba/F-BCR/ABL T315I (Imatinib-resistant) (FIG. 3B) cells were seeded in a 96-well plate at 3.5×10³ cells/well in the presence of the indicated concentrations of Imatinib or rapamycin (5 nM) alone or in combination. Cell proliferation was measured after 48 hr of drug treatment. Values represent the means for triplicate determinations; bars ±SD (see FIGS. 3A and 3B).

As in the other cell systems (FIGS. 1, 2), rapamycin (5 nM) inhibited the proliferation of Ba/F-BCR/ABL WT cells (˜60% after 48 hours of exposure). Likewise, Imatinib potently inhibited the proliferation of these cells, and combining the two agents resulted in enhanced inhibition (FIG. 3A).

Rapamycin alone showed comparable inhibition of proliferation of Ba/F3 cells expressing BCR/ABL WT and BCR/ABL T315I (FIGS. 3A,B), as would be predicted if mTOR were a critical downstream effector of BCR/ABL. Consistent with the Imatinib resistance of the T315I mutant, doses of Imatinib (0.5-1 μM) that inhibited proliferation of Ba/F-BCR/ABL WT cells by more than 50% caused little or no inhibition of Ba/F-BCR/ABL T315I cells (FIG. 3B).

Remarkably, however, combining low dose rapamycin with Imatinib markedly enhanced the growth inhibitory effect of Imatinib on Ba/F-BCR/ABL T315I cells (FIG. 3B). These results suggest that combining rapamycin with Imatinib may be useful in treating Imatinib-resistant CML.

### Example 4

### Immunoblotting BCR/ABL-Expressing Cells with Phospho-Specific Antibodies

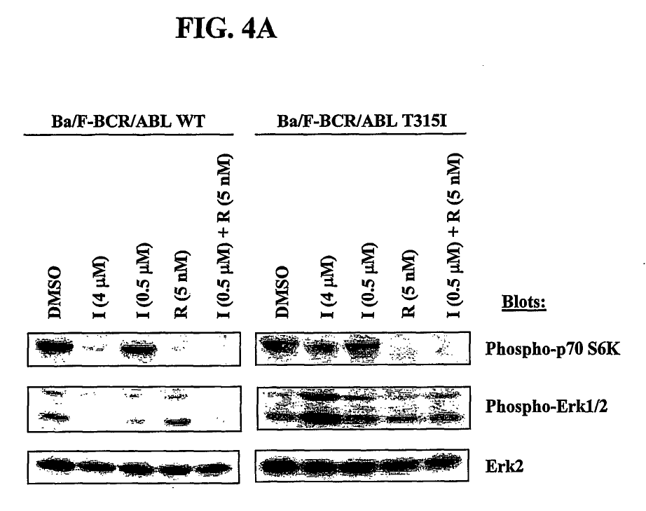

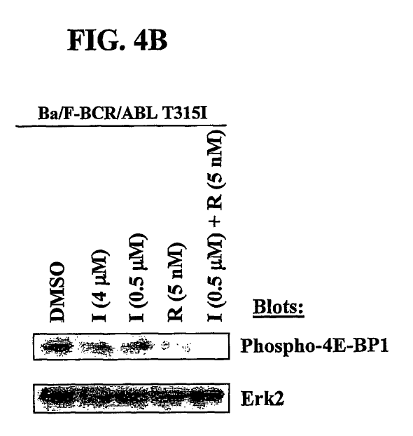

Ba/F-BCR/ABL WT and Ba/F-BCR/ABL T315I cells were incubated with the indicated concentrations (see FIG. 4A) of Imatinib/Rapamycin for 18 hours. Cell lysates were prepared and equal amounts (50 μg) of protein were resolved by SDS-PAGE followed by immunoblot analysis with the indicated phospho-specific antibodies. To control for loading, the blots were stripped and reprobed with anti-Erk2 antibodies.

The effects of Imatinib and Rapamycin on 4E-BP1 phosphorylation were determined in Ba/F-BCR/ABL T315I cells by immunoblotting with phospho-4E-BP1 antibodies. Erk2 was used a loading control (see FIG. 4B).

Treatment of Ba/F-BCR/ABL WT cells with a low dose of Imatinib (0.5 μM), which causes ˜70% inhibition of proliferation (FIG. 4A), resulted in partial inhibition of p70 S6K activation (FIG. 4B). Not surprisingly, the same dose failed to inhibit p70 S6K in Imatinib-resistant Ba/F-BCR/ABL T315I cells. At higher doses of Imatinib (4 μM), which completely inhibit Ba/F-BCR/ABL WT proliferation, p70 S6K activation was nearly completely inhibited as well, whereas in Ba/F-BCR/ABL T315I cells, which show only partial inhibition of proliferation at this dose, p70 S6K was inhibited by only 50%. Thus, the partial inhibition of proliferation in Ba/F-BCR/ABL T315I cells treated with 4 μM Imatinib was paralleled by partial inhibition of p70 S6K activation. However, rapamycin at doses between 0.5-5 nM almost completely inhibited activation of p70 S6K in both Ba/F-BCR/ABL WT and Ba/F-BCR/ABL T315I cells (FIG. 4A). Interestingly, 4E-BP1 phosphorylation displayed more resistance to treatment with either Imatinib or rapamycin than p70 S6K. Treatment of Ba/F-BCR/ABL T315I cells with Imatinib (0.5-4 μM) or rapamycin (5 nM) alone caused partial inhibition of 4E-BP1phosphorylation (FIG. 4B); at the same dose, rapamycin treatment totally inhibited p70 S6K phosphorylation. Combining Imatinib with rapamycin led to complete inhibition of 4E-BP1phosphorylation in Ba/F-BCR/ABL T315I cells (FIG. 4B). As expected, rapamycin treatment did not inhibit Erk activation in either of these cell lines. Although Imatinib potently inhibited Erk activation in Ba/F-BCR/ABL WT cells, it failed to inhibit Erk activation in Imatinib-resistant cells (FIG. 4A). Indeed, Erk activation actually increased in Ba/F-BCR/ABL T315I cells at higher doses of Imatinib. The reason for the paradoxical effect of high doses of Imatinib in these cells is unclear, although similar results were obtained in studies of Imatinib-resistant K562 cells treated with Imatinib (Yu et al., Cancer Res. 62:188 (2002)).

Taken together, our results indicate that sub-therapeutic doses of Imatinib (e.g., low dose treatment of Ba/F-BCR/ABL WT cells or high dose treatment of Imatinib-resistant cells), leave the p70 S6K arm of the mTOR pathway partially active and thus susceptible to further inhibition by rapamycin.

### Example 5

### Effects of PKC412 and Rapamycin on Proliferation of Ba/F-FLT3-ITD Cells

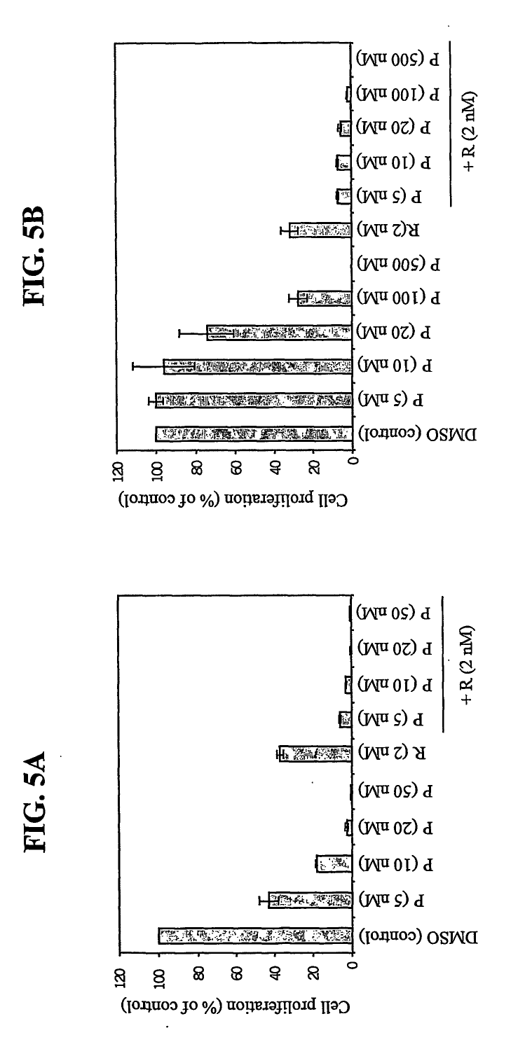

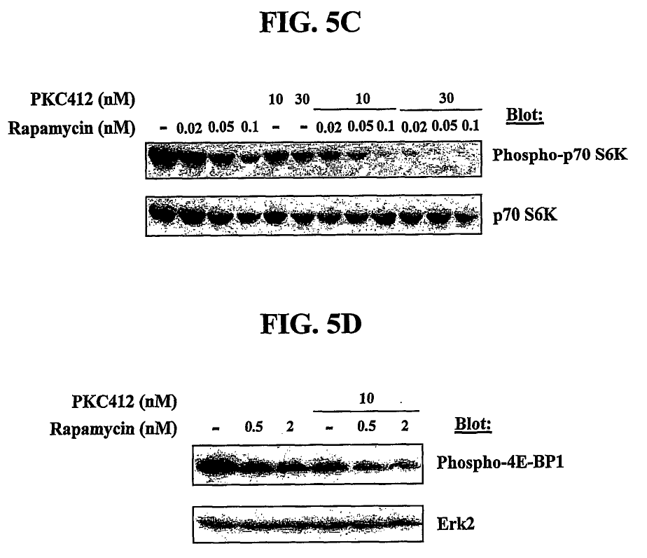

PKC412-sensitive Ba/F-FLT3-ITD (FIG. 5A) and PKC412-resistant Ba/F-FLT3-ITD F6911 (FIG. 5B) cells were exposed to the indicated concentrations of PKC412 for 48 hours in the presence or absence of 2 nM rapamycin, after which cell proliferation was measured by [³H]-thymidine incorporation. Values represent the means for triplicate determinations; bars ±SD (see FIGS. 5A and 5B).

As expected, PKC412 inhibited the proliferation of Ba/F-FLT3-ITD cells in a dose-dependent manner. Moreover, whereas PKC412 (5 nM) or rapamycin (2 nM) alone caused approximately 55-60% inhibition, combining these drugs led to more than 94% inhibition of proliferation of Ba/F-FLT3-ITD cells (FIG. 5A). We also tested the effects of the rapamycin/PKC412 combination on PKC412-resistant Ba/F-FLT3-ITD cells. A PKC412-resistant cell line was derived by transduction of a PKC412-resistant FLT3-ITD F691I mutant into Ba/F3 cells. These cells also exhibit significantly higher expression of FLT3-ITD F691I protein compared to Ba/F-FLT3-ITD cells. As observed with rapamycin treatment of Imatinib-resistant BCR/ABL mutants, PKC412-resistant Ba/F-FLT3-ITD F61I cells remained sensitive to the growth inhibitory effects of rapamycin alone, but were resistant to doses of PKC412 as high as 20 nM (FIG. 5B). However, rapamycin (2 nM) combined with PKC412 (5 nM) dramatically inhibited the proliferation of Ba/F-FLT3-ITD F691I cells (>93%, FIG. 5B).

PKC412-sensitive Ba/F-FLT3-ITD cells were treated with the indicated concentrations of rapamycin or PKC412 or both, after which cell lysates were subjected to immunoblotting with phospho-specific p70 S6 K (FIG. 5C) and 4E-BP1 (FIG. 5D) antibodies. The blots were reprobed for p70 S6 kinase and Erk2, respectively, to ensure equivalent loading.

Similar to the effects of the Imatinib/rapamycin combination on BCR/ABL-expressing cells, treatment of Ba/F-FLT3-ITD cells with the combination of PKC412 and rapamycin led to a greater decrease in phosphorylation of p70 S6K. Likewise, 4E-BP1 phosphorylation was >10 fold more resistant to rapamycin than p70 S6K in these cells (FIGS. 5C and 5D), and could only be substantially inhibited by the two-drug combination.

### Example 6

### Addition of an MEK Inhibitor (UO126) Increases the Inhibitory Effects of Rapamycin/Imatinib and Rapamycin/PKC412 combinations

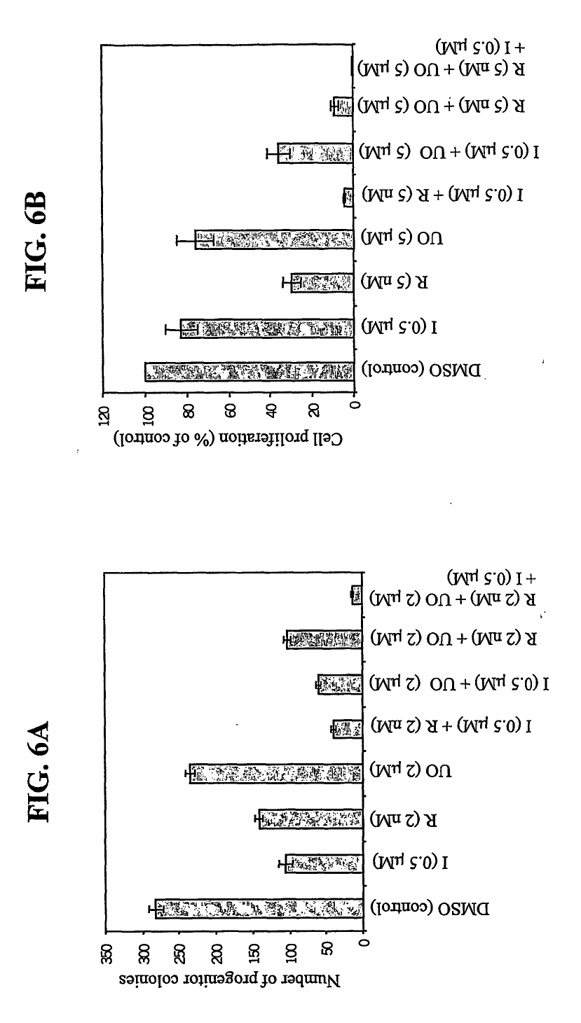

Bone marrow cells from mice were transduced with BCR/ABL-expressing retroviruses. Imatinib (0.5 μM), rapamycin (2 nM) or UO126 (2 μM) alone or in various combinations, as indicated, were added to the transduced bone marrow cells prior to plating for myeloid colony assays. The number of colonies from triplicate platings was determined after 10 days (see FIG. 6A).

Low doses of rapamycin (2 nM or Imatinib (0.5 μM) alone inhibited BCR/ABL-evoked myeloid colony formation by ˜50-60%, whereas low dose UO126 (2 μM) was only slightly (16%) inhibitory (FIG. 6A). UO126 plus rapamycin had an additive effect, whereas UO126 plus Imatinib or rapamycin plus Imatinib synergistically inhibited myeloid colony outgrowth (FIG. 6A). Remarkably, however, a combination of low doses of all three agents caused profound inhibition (96%) in this assay (FIG. 6A).

Imatinib-resistant Ba/F-BCR/ABL T315I cells were exposed to the indicated concentrations of Imatinib, rapamycin or UO126 alone or in various combinations for 48 hours, followed by measurement of cell proliferation by [³H]-thymidine incorporation (see FIG. 6B).

UO126 (at 5 μM) or Imatinib (at 0.5 μM) alone exhibited only modest inhibitory effects on the proliferation of Ba/F-BCR/ABL T315I cells, although rapamycin (5 nM) alone caused about 70% inhibition of proliferation of these cells (FIG. 6B). UO126 plus Imatinib showed additive inhibitory effects, whereas UO126 plus rapamycin or Imatinib plus rapamycin greatly enhanced inhibition of Ba/F-BCR/ABL T315I cell proliferation (FIG. 6B). Again, however, the triple combination (rapamycin plus UO126 plus Imatinib) caused even more robust inhibition of Ba/F-BCR/ABL T315I cells.

PKC412-resistant Ba/F-FLT3-ITD F691I cells were incubated with the indicated concentrations of PKC412 or rapamycin or UO126 alone or in combination for 48 hours. Cell proliferation was measured by a [³H]-thymidine incorporation assay (see FIG. 6C).

Whereas PKC412 at 10 nM had almost no effect, rapamycin (2 nM) or UO126 (5 μM ) treatment inhibited PKC412-resistant Ba/F-FLT3-ITD cell proliferation by 68% or 40%, respectively. Moreover, the proliferation of PKC412-resistant Ba/F-FLT3-ITD F691I cells was profoundly inhibited when UO126 was added in combination with rapamycin (FIG. 6C). Rapamycin plus PKC412 also had synergistic inhibitory effects, whereas UO126 plus PKC412 exhibited additive effects on the proliferation of PKC412-resistant Ba/F-FLT3-ITD F691I cells. The three-drug combination (rapamycin plus PKC412 plus UO126) almost completely (>99%) inhibited the growth and survival of PKC412-resistant Ba/F-FLT3-ITD F691I cells.

### Example 7

### Effects of Imatinib, Rapamycin, and UO126 Combinations on Cell Cycle Distribution of BCR/ABL-Transformed B-Lymphoid Cells

3 million cells in media as described above and treated with the indicated drugs or left untreated for 24 hours, were fixed, stained with propidium iodide and subject to flow cytometry for cell cycle analysis. Results are provided in Table 4.

Decreased [³H]-thymidine uptake could reflect a decreased rate of cell cycle progression and/or an increase in cell death. Treatment of BCR/ABL-transformed B lymphoblasts with low doses of Imatinib (1 μM) alone led to cell cycle arrest (Table 4); higher (therapeutic) doses were cytotoxic as expected (data not shown). Rapamycin (10 nM) alone caused G1 arrest, but no significant apoptosis. However, the combination of Imatinib (1 μM ) and rapamycin (10 nM) evoked a significant increase in apoptosis, as indicated by sub-G1 DNA content (Table 4).

Whereas low doses of either agent alone caused G₁ arrest, cells exposed to Imatinib/rapamycin exhibited substantial apoptosis (Table 4). This “gain of function” argues strongly that the drug combination has synergistic inhibitory effects.

TABLE 4
Effects of Imatinib, Rapamycin, andUO126 Combinations
on Cell Cycle Distribution of BCR/ABL-transformed
primary B-lymphoblast cells

| | % of viable cells | | | |
|---|---|---|---|---|
| Drugs Treated | in phase: | | | Total % |
| Cells | G₁ | S | G₂/M | Apoptotic |
| DMSO (control) | 50.6 | 44.7 | 4.7 | 3.9 |
| Imatinib (1 μM) | 80.9 | 16 | 3.1 | 6.7 |
| Rapamycin (10 nM) | 79.9 | 18.3 | 1.8 | 8.6 |
| Rapamycin (10 nM) + Imatinib | 83.4 | 16.6 | 0 | 16.5 |
| (1 μM) | | | | |
| UO126 (5 μM) | 44.6 | 51.6 | 3.8 | 4.6 |
| UO126 (5 μM) + Imatinib | 83.2 | 15.2 | 1.6 | 6.6 |
| (1 μM) | | | | |

### Example 8

### Effects of Herceptin™ and Rapamycin on Neu-Low and Neu-High Cell Lines

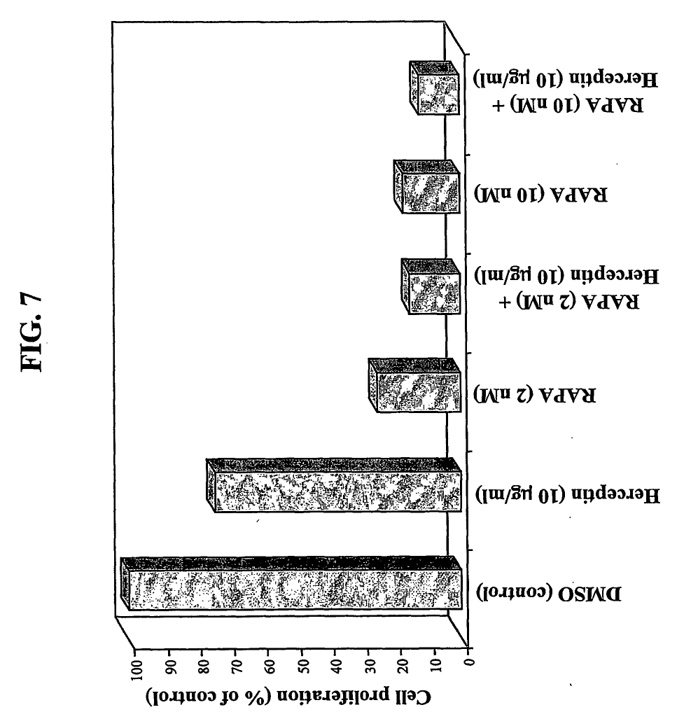

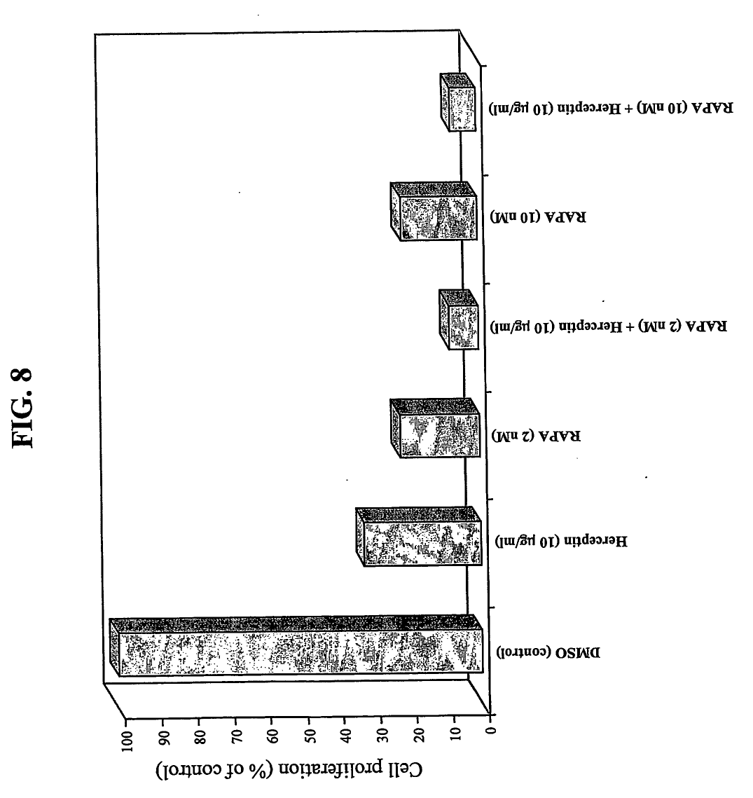

MCF7 (Neu-low) and SKBR3 (Neu-high) were seeded at 30,000 cells per 6-well cluster (35 mm plate). After 24 hours, cells were treated with herceptin (10 mcg/ml) plus/minus the indicated concentrations of rapamycin. Media was changed every 2 days with fresh drugs. After 7 days, cell number was determined by Coulter Counter (see FIGS. 7 and 8).

This data shows that combining rapamycin with herceptin greatly enhances inhibitory effects on proliferation of breast cancer cells.

### Example 9

### Effects of Rapamycin and/or Imatinib on PTEN-Positive and PTEN-Negative Cell Lines

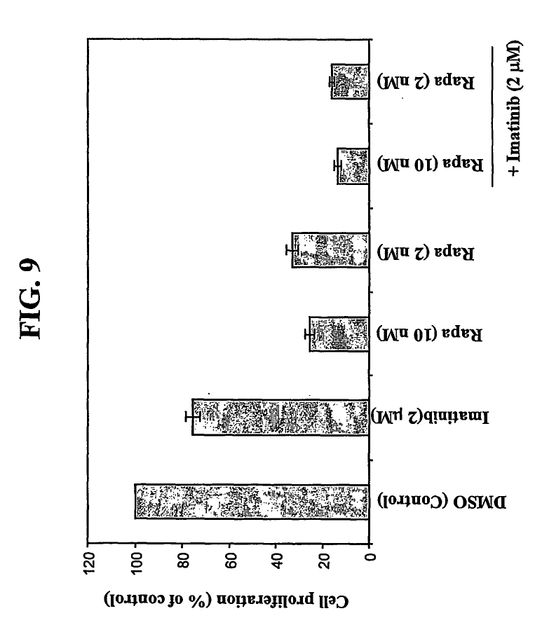

PTEN-positive human prostate cancer cells (CWR22) were seeded at 30,000 cells per 6-well cluster (35 mm plate). After 24 hours, cells were treated with Imatinib (2 μg/ml) plus/minus the indicated concentrations of rapamycin. Media was changed every 2 days with fresh drugs. After 7 days, cell number was determined by hemocytometer (see FIG. 9).

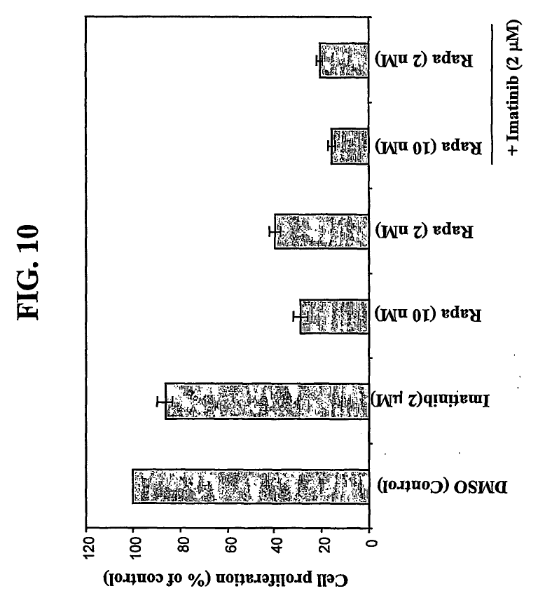

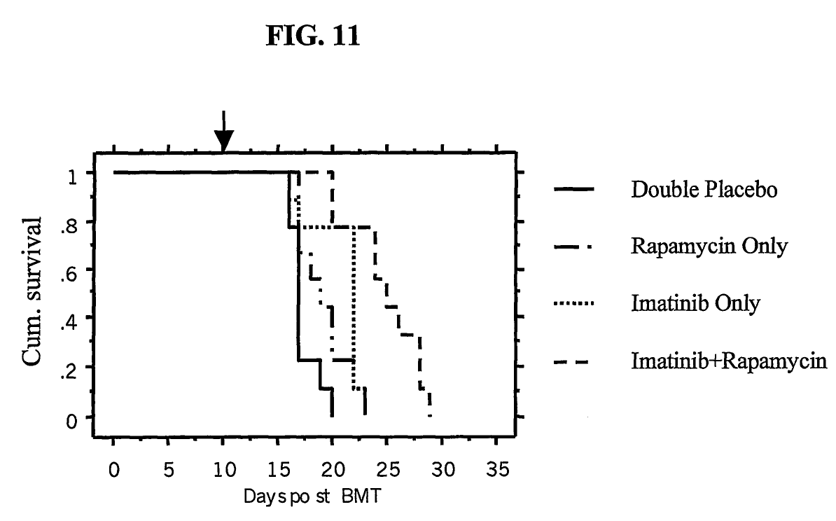

PTEN-negative human prostate cancer cells (LnCaP) were seeded at 30,000 cells per 6-well cluster (35 mm plate). After 24 hours, cells were treated with Imatinib (2 μg/ml) plus/minus the indicated concentrations of rapamycin. Media was changed every 2 days with fresh drugs. After 7 days, cell number was determined by hemocytometer (see FIG. 10).

Rapamycin alone has significant inhibitory effect on proliferation of both PTEN-positive and PTEN-negative prostate cancer cells. Combining rapamycin with Imatinib, which inhibits PDGFR, enhances the antiproliferative effect upon the growth of prostate cancer cells.

### Example 11

### Rapamycin/Imatinib Combinations Improve Survival in Mouse Model CML

BALB/C donor mice were primed with intraperitoneal injection of 5′ fluorouracil (150 mg/kg). After 5 days, bone marrow cells were collected and transduced with MSCV p210 (BCR/ABL)-IRES-GFP viruses by two rounds of spinfection. After second round of transduction, cells were resuspended in Hanks balanced salt solution and injected (1×10⁶ cells/0.5 ml) into the lateral tail vein of lethally irradiated (2×450 cGy) female recipient BALB/C mice. The trial design consisted of four groups (placebo/placebo; placebo/rapamycin; Imatinib/placebo; Imatinib/rapamycin), each group containing 9 mice. Rapamycin was administered at a dose of 7.0 mg/kg/day and Imatinib at a dose of 70 mg/kg/day. Imatinib and its placebo were administered by oral gavage, whereas rapamycin and its placebo were administered via introperitoneal injection. All animals in the trial received two gavage treatments and one IP treatment per 24 hours. Treatment was started from day 9 after bone marrow transplantation (BMT) and continued until the mice died. The log rank statistics were used to attach a significance level to the difference in the survival curves.

At the time of death of the first double placebo animal (day 20), one mouse per group was sacrificed for full analysis. These mice were censored from the statistical analysis of survival. Peripheral blood was collected from the retroorbital cavity using a heparinized glass capillary. Blood smears were stained with Wright and Giemsa. Manual and automated (ADIVA 120 Hematology system, Bayer) total and differential blood cell counts were performed, as well as histopathologic exam of relevant organs (spleen, liver, heart, lungs, intestine, hindlimb bones, and kidneys). Preparation of single-cell suspensions from spleen and bone marrow for flow cytometry was performed as described previously (Schwaller et al, Embo J 17:5321 (1998), Kelly and Weisberg et al, Cancer Cell 1:433 (2002)).

This study validates the previous findings that imatinib treatment increases survival in the murine BCR/ABL disease model, even at a sub-optimal dose. As expected, animals treated with imatinib plus placebo showed better survival than those treated with double placebo (p=0.002). This trial also showed a protective effect of rapamycin alone against BCR/ABL disease. Mice treated with rapamycin plus placebo survived longer than those treated with double placebo (p=0.04). Finally, animals treated with both imatinib and rapamycin showed a statistically significant improvement in survival when compared to those treated with either imatinib alone (p=0.003) or rapamycin alone, (p=0.0003).

### Other Embodiments

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology or related fields are intended to be within the scope of the invention.

1. A method of treating a neoplasm characterized by abnormally high levels of tyrosine kinase activity in a patient in need thereof, said method comprising administering to said patient at least one mTOR inhibitor together or in parallel with at least one tyrosine kinase inhibitor in amounts that together are effective to treat said neoplasm.

2. The method of claim 1, wherein said mTOR inhibitor is a rapamycin macrolide.

3. The method of claim 1, wherein said rapamycin macrolide is, rapamycin, CCI-779, Everolimus, or ABT-578.

4. The method of claim 1, wherein said tyrosine kinase inhibitor is selected from the group consisting of a small molecule inhibitor, an antibody, an antisense oligomer, and an RNAi inhibitor.

5. The method of claim 4, wherein said small molecule inhibitor is selected from the group consisting of Imatinib, SU101, ZD1839, OSI-774, CI-1033, SU5416, SU6668, ZD4190, ZD6474, PTK787, PKI166, GW2016, EKB-509, EKB-569, CEP-701, CEP-751, PKC412, SU11248, and MLN518.

6. The method of claim 4, wherein said antibody is selected from the group consisting of trastuzumab, C225, rhu-Mab VEGF, MDX-H210, 2C4, MDX-447, IMC-1C11, EMD 72000, RH3, and ABX-EGF.

7. The method of claim 1, further comprising administering an MEK inhibitor.

8. The method of claim 7, wherein said MEK inhibitor is selected from PD184352, PD198306, PD98059, UO126, Ro092210, and L783277.

9. The method of claim 1, wherein said neoplasm is selected from carcinoma of the bladder, breast, colon, kidney, liver, lung, head and neck, gall-bladder, ovary, pancreas, stomach, cervix, thyroid, prostate, or skin; a hematopoietic tumor of lymphoid lineage; a hematopoietic tumor of myeloid lineage; a tumor of mesenchymal origin; a tumor of the central or peripheral nervous system; melanoma; seminoma; teratocarcinoma; osteosarcoma; thyroid follicular cancer; and Kaposi's sarcoma.

10. The method of claim 9, wherein said hematopoietic tumor of lymphoid lineage is selected from leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma.

11. The method of claim 9, wherein said hematopoietic tumor of myeloid lineage is selected from acute myelogenous leukemia, chronic myelogenous leukemia, multiple myelogenous leukemia, myelodysplastic syndrome and promyelocytic leukemia.

12. The method of claim 9, wherein said tumor of mesenchymal origin is fibrosarcoma or rhabdomyosarcoma.

13. The method of claim 9, wherein said tumor of the central or peripheral nervous system is selected from astrocytoma, neuroblastoma, glioma and schwannomas.

14. The method of claim 2, wherein said tyrosine kinase activity is epidermal growth factor receptor activity; said neoplasm is selected from non-small-cell lung cancer, breast cancer, ovarian cancer, bladder cancer, prostate cancer, salivary gland cancer, pancreatic cancer, endometrial cancer, colorectal cancer, kidney cancer, head and neck cancer, and glioblastoma multiforme; and said tyrosine kinase inhibitor is selected from the group consisting of SU101, ZD1839, OSI-774, CI-1033, PKI166, GW2016, EKB-509, EKB-569, trastuzumab, C225, MDX-H210, 2C4, MDX-447, and ABX-EGF.

15. The method of claim 2, wherein said tyrosine kinase activity is human epidermal growth factor receptor-2 activity; said neoplasm is selected from the group consisting of breast cancer, ovarian cancer, bladder cancer, salivary gland cancer, endometrial cancer, pancreatic cancer, and non-small-cell lung cancer; and said tyrosine kinase inhibitor is selected from the group consisting of CI-1033, GW2016, trastuzumab, MDX-H210, MDX-447, ABX-EGF, EMD 72000, RH3, and 2C4.

16. The method of claim 15, wherein said tyrosine kinase inhibitor is trastuzumab.

17. The method of claim 2, wherein said tyrosine kinase activity is platelet derived growth factor receptor activity; said neoplasm is selected from the group consisting of gastrointestinal stromal tumor, small cell lung cancer, glioblastoma multiforme, and prostate cancer; and said tyrosine kinase inhibitor is selected from the group consisting of Imatinib, SU101, MLN518, and PTK787.

18. The method of claim 17, wherein said wherein said tyrosine kinase inhibitor is Imatinib.

19. The method of claim 2, wherein said tyrosine kinase activity is Flt-3 activity; said neoplasm is acute myeloid leukemia and said tyrosine kinase inhibitor is selected from MLN518, SU11248, and PKC412.

20. The method of claim 19, wherein said tyrosine kinase inhibitor is PKC412.

21. The method of claim 2, wherein said tyrosine kinase activity is tropomyosin receptor kinase activity; said neoplasm is prostate cancer or pancreatic cancer; and said tyrosine kinase inhibitor is Imatinib, CEP701 or CEP705.

22. The method of claim 2, wherein said tyrosine kinase activity is BCR/ABL activity; said neoplasm is chronic myelogenous leukemia or acute lymphoblastic leukemia; and said tyrosine kinase inhibitor is Imatinib.

23. The method of claim 2, wherein said tyrosine kinase is a vascular endothelial growth factor receptor kinase; said cancer is any solid tumor; and said tyrosine kinase inhibitor is selected from the group consisting of SU5416, SU6668, ZD4190, ZD6474, PTK787, IMC-1C11, and rhu-Mab VEGF.

24. The method of claim 1, wherein said neoplasm is resistant to said tyrosine kinase inhibitor.

25. (canceled)

26. (canceled)

27. The method of claim 1, wherein said mTOR inhibitor and said tyrosine kinase inhibitor are administered in parallel within 30 days of each other.

28. The method of claim 27, wherein said mTOR inhibitor and said tyrosine kinase inhibitor are administered in parallel within 5 days of each other.

29. The method of claim 28, wherein said mTOR inhibitor and said tyrosine kinase inhibitor are administered in parallel within 24 hours of each other.

30. The method of claim 1, wherein said rapamycin macrolide and said tyrosine kinase inhibitor are administered together.

31. (canceled)

32. (canceled)

33. A method of treating leukemia in a patient in need thereof, said method comprising administering rapamycin to said patient in an amount effective to treat said leukemia.

34. A method of treating a neoplasm in a patient in need thereof, said method comprising administering to said patient at least one mTOR inhibitor together or in parallel with at least one tyrosine kinase inhibitor and at least one MEK inhibitor in amounts that together are effective to treat said neoplasm.

35. The method of claim 34, wherein said neoplasm is selected from carcinoma of the bladder, breast, colon, kidney, liver, lung, head and neck, gall-bladder, ovary, pancreas, stomach, cervix, thyroid, prostate, or skin; a hematopoietic tumor of lymphoid lineage; a hematopoietic tumor of myeloid lineage; a tumor of mesenchymal origin; a tumor of the central or peripheral nervous system; melanoma; seminoma; teratocarcinoma; osteosarcoma; thyroid follicular cancer; and Kaposi's sarcoma.

36. The method of claim 34, wherein said mTOR inhibitor is selected from rapamycin, CCI-779, Everolimus, and ABT-578.

37. The claim 34, wherein said tyrosine kinase inhibitor is selected from Imatinib, SU101, ZD1839, OSI-774, CI-1033, SU5416, SU6668, ZD4190, ZD6474, PTK787, PKI166, GW2016, EKB-509, EKB-569, CEP-701, CEP-751, PKC412, SU11248, MLN518, trastuzumab, C225, rhu-Mab VEGF, MDX-H210, 2C4, MDX-447, IMC-1C11, EMD 72000, RH3, and ABX-EGF.

38. The method of claim 34, wherein said MEK inhibitor is selected from PD184352, PD198306, PD98059, UO126, Ro092210, and L783277.

39-87. (canceled)
